(19) United States
(12) Patent Application Publication
(10) Pub. No.: US 2026/0035417 A1
(43) Pub. Date: Feb. 5, 2026

(54) USE OF AN ANNELID MOLECULE FOR TREATING AND/OR PREVENTING AT LEAST ONE COMPLEMENT-ACTIVATION ASSOCIATED DISEASE

(71) Applicants: HEMARINA, Morlaix (FR); INSTITUT NATIONAL DE LA SANTE ET DE LA RECHERCHE MEDICALE, Paris (FR); UNIVERSITE TOULOUSE III-PAUL SABATIER, Toulouse (FR); CENTRE NATIONAL DE LA RECHERCHE SCIENTIFIQUE, Paris (FR); CENTRE HOSPITALIER UNIVERSITAIRE DE TOULOUSE, Toulouse (FR)

(72) Inventors: Franck ZAL, Morlaix (FR); Yves RENAUDINEAU, Toulouse (FR); Bénédicte BRANO, Vieille Toulouse (FR)

(21) Appl. No.: 18/997,382

(22) PCT Filed: Jul. 25, 2023
(86) PCT No.: PCT/EP2023/070537

§ 371 Date: Jan. 21, 2025

(30) Foreign Application Priority Data
Jul. 26, 2022 (EP) 22306111.0

(51) Int. Cl. C07K 14/435; A61K 38/00

(52) U.S. Cl. CPC C07K 14/43536; A61K 38/00

(57) ABSTRACT

The present invention concerns the use of a molecule chosen from a globin, a globin protomer or an extracellular hemoglobin of Annelids, for treating and/or preventing at least one complement-activation associated disease, preferably ischemia/reperfusion injury and/or transplant rejection.

The present invention concerns the use of a molecule chosen from a globin, a globin protomer or an extracellular hemoglobin of Annelids, for treating and/or preventing at least one complement-activation associated disease, preferably ischemia/reperfusion injury and/or transplant rejection.

Kidney transplantation is the best therapy in presence of chronic kidney disease. Renal biopsy gives several clinical information about the graft and its prognosis. Renal transplant recipients may suffer from degenerative lesions denominated Chronic Allograft Nephropathy and ultimately from graft rejections, such as T cell-mediated rejection (TCMR) and antibody-mediated rejection (ABMR). Renal graft rejections, including TCMR and ABMR, are usually diagnosed on the basis of histologic evaluation performed in response to biochemical evidence of graft impairment (e.g., elevated creatinine levels). ABMR is a clinical and histopathologic diagnosis based on detection of allograft dysfunction with evidence of endothelial inflammation, and it is mediated by circulating antibodies directed against donor antigens in the allograft. Specifically, the pathology is caused by binding of the recipient's antibodies to human leukocyte antigens (HLA) expressed on endothelial cells of the transplanted organ. Said antibodies subsequently activate the classical pathway of complement.

Ischemia-reperfusion injury (I/R) is the tissue damage caused when blood supply returns to tissue after a period of ischemia. Reestablishment of blood flow is essential to salvage ischaemic tissues. However, reperfusion itself paradoxically causes further damage, threatening function and viability of the organ. Numerous studies have shown that complement is activated after I/R, although the mechanisms may vary between different organs. Complement activation in the ischemic heart and intestine may be initiated by immunoglobulin, but it primarily involves the lectin pathway. In the kidney, complement activation primarily involves the alternative pathway.

Complement activation is an important cause of tissue injury in patients with transplanted organs. Indeed, the complement pathway is a key element of the innate immune system composed of multiple proteins, and needs to be activated in order to contribute to the elimination of invading pathogens, apoptotic or necrotic cells and immune complexes. Complement activation can be initiated by immunoglobulins (classical pathway) or by lectins (lectin pathway) leading to the formation of the complement 1 (C1) complex (C1qrs) and its associated proteases C4 and C2. In addition, the spontaneous hydrolysis of the C3 protein in a fluid phase, together with the activation of the factors B and D, initiate the alternative pathway and leads to the binding of C3b on a foreign surface. Once activated, the three pathways converge to a common pathway at C3 to form the C3 convertases (C4bC2a or C3bBb) and progress to the formation of C5 convertases (C4bC2a3b or C3bBb3b) that are necessary for the constitution of the membrane attack complex (MAC, C5b9) on the target membrane. During C3 and C5 convertase formation, functional activated fragments are generated including the anaphylatoxins C3a and C5a that attract phagocytic cells and the antigen bound C3d derived from bound C3b that regulates in a feedback loop the antibody (Ab) response.

Accordingly, and from a therapeutic point of view, selective inhibition of the complement pathway is a promising strategy in transplantation to prevent ischemia/reperfusion injury (via the alternative pathway) and/or antibody-mediated allograft rejection (via the classical pathway). In other words, selective inhibition of the complement pathway is a promising strategy for increasing the success rates of transplantations.

There is thus a need for an efficient way of preventing ischemia/reperfusion injury and/or antibody-mediated rejection after transplantation. There is also a need for selectively inhibiting the complement pathway, thereby providing efficient and reliable means for preventing and/or decreasing transplant rejections.

The present invention solves this need. Indeed, as shown in the examples, the inventors tested and observed an interference of the extracellular hemoglobin of Arenicola marina (M101) on the complement-dependent cytotoxicity crossmatch (CDC-XM) reaction. Moreover, they found that M101 was effective, at concentrations used in the graft preservation solution (1-5 g/L), to inhibit C3 convertase, which may be responsible for the better recovery of renal function.

Thus, the present invention relates to the use of a molecule chosen from a globin, a globin protomer, or an extracellular hemoglobin of Annelids, for treating and/or preventing at least one complement-activation associated disease.

Preferably, the present invention relates to the use of a molecule chosen from a globin, a globin protomer, or an extracellular hemoglobin of Annelids, for treating and/or preventing ischemia/reperfusion injury and/or transplant rejection such as antibody-mediated rejection.

Preferably, said molecule chosen from a globin, a globin protomer, or an extracellular hemoglobin of Annelids is a C3 convertase inhibitor. Preferably the C3 convertase is C3bBb or C4bC2a.

Preferably, said molecule chosen from a globin, a globin protomer, or an extracellular hemoglobin of Annelids is an inhibitor of C3bBb and/or C4bC2a.

Preferably, the molecule chosen from a globin, a globin protomer, or an extracellular hemoglobin of Annelids of the invention, allows treating and/or preventing at least one complement-activation associated disease, by inhibiting the C3 convertases.

Thus, preferably, the molecule chosen from a globin, a globin protomer, or an extracellular hemoglobin of Annelids of the invention, allows treating and/or preventing ischemia/reperfusion injury, and/or preventing transplant rejection such as antibody-mediated rejection, by inhibiting the C3 convertases.

Preferably, it inhibits C4bC2a and/or C3bBb.

By “complement-activation associated disease”, it is meant a disease that is caused by complement activation. The complement-activation associated disease is preferably chosen from immune and autoimmune disorders such as systemic lupus erythematosus, anti-neutrophil cytoplasmic antibody (ANCA)-associated vasculitis and Crohn's disease; renal diseases such as C3 glomerulopathies, atypical hemolytic-uremic syndrome and other thrombotic microangiopathies; acute and chronic inflammation dysfunctions such as COVID-19 and other viral infections, hemodialysis, implants, sepsis and cancer; age-related and degenerative diseases such as Alzheimer's syndrome, schizophrenia and macular degeneration; pregnancy-related complications such as preeclampsia; diseases involving ischemia such as ischemia/reperfusion injury, transplant rejection such as antibody-mediated rejection, heart and vascular diseases (for example ischemia/myocardial infarction), cerebral and intestine embolism (for example stroke or mesenteric ischemia); and hemolytic disorders such as paroxysmal nocturnal hemoglobinuria.

Preferably the complement-activation associated disease is chosen from diseases involving ischemia, more preferably from ischemia/reperfusion injury, transplant rejection such as antibody-mediated rejection, heart and vascular diseases (for example ischemia/myocardial infarction), cerebral and intestine embolism (for example stroke or mesenteric ischemia). More preferably the complement-activation associated disease is chosen from ischemia/reperfusion injury and transplant rejection such as antibody-mediated rejection.

As indicated above, ischemia-reperfusion injury (I/R) is the tissue damage caused when blood supply returns to tissue after a period of ischemia. The treatment of I/R in a given organ allows restoring the functionality of said organ, and/or decreasing and/or inhibiting the deleterious effects of said ischemia on the organ.

The term “transplantation” refers to the insertion of a transplant (also called graft) into a recipient, whether the transplantation is syngeneic (where the donor and recipient are genetically identical), allogeneic (where the donor and recipient are of different genetic origins but of the same species) or xenogeneic (where the donor and recipient are from different species). Thus, in a typical scenario, the host is human and the graft is an isograft, derived from a human of the same or different genetic origins. In another scenario, the graft is derived from a species different from that into which it is transplanted, including animals from phylogenically widely separated species. Transplantation may also refer to medical devices, such as implanted mechanical supports or implants (for example breast implants), which may be inserted into a recipient.

The transplant may be any organ, preferably a kidney, a heart, a liver, an intestine, a pancreas or a lung. Preferably it is a kidney.

As used herein, the terms “kidney graft”, “renal graft”, “kidney transplant” or “renal transplant” are used herein interchangeably and refer to the organ (i.e. the kidney) which is transplanted to a patient suffering from End-Stage Renal Disease (ESRD).

As used herein, the terms “transplant rejection” or “graft rejection” encompass both acute and chronic transplant rejection.

“Acute transplant rejection” is the rejection by the immune system of a tissue transplant recipient when the transplanted tissue is immunologically foreign. Acute transplant rejection is characterized by infiltration of the transplant tissue by immune cells of the recipient, which carry out their effector function and destroy the transplant tissue. The onset of acute rejection is rapid and generally occurs in humans within a few weeks after transplant surgery. Generally, acute transplant rejection can be inhibited or suppressed with immunosuppressive drugs such as rapamycin, cyclosporin and the like.

“Chronic transplant rejection” generally occurs in humans within several months to years after engraftment, even in the presence of successful immunosuppression of acute rejection. Fibrosis is a common factor in chronic rejection of all types of organ transplants. Preferably, the transplant rejection is an acute transplant rejection or a chronic transplant rejection.

Preferably, the transplant rejection is an antibody-mediated rejection (ABMR) or a T cell-mediated rejection (TCMR).

Preferably, the transplant rejection is an acute ABMR or a chronic ABMR.

Preferably, the transplant rejection is an acute TCMR or a chronic TCMR.

The molecule chosen from a globin, a globin protomer, or an extracellular hemoglobin of Annelids of the invention is an oxygen transporter. “Oxygen transporter” means any molecule capable of reversibly transporting oxygen from the environment to target cells, tissues or organs. The oxygen transporter according to the invention comes from Annelids, which are marine invertebrate animals.

The extracellular hemoglobin of Annelids is present in the three classes of Annelids: Polychetes, Oligochaetes and Achetes. We talk about extracellular hemoglobin because it is naturally not contained in a cell, and may, therefore, circulate freely in the blood system without the need for chemical modification to stabilize or make it functional.

The extracellular hemoglobin of Annelids is a giant biopolymer with a molecular weight between 2000 and 4000 kDa, consisting of approximately 200 polypeptide chains between 4 and 12 different types, which are generally grouped into two categories.

The first category, comprising 144 to 192 elements, groups together the so-called “functional” polypeptide chains which carry an active heme-type site and are capable of reversibly binding oxygen; these are globin-type chains (eight types in total for Arenicola marina hemoglobin: a1, a2, b1, b2, b3, c, d1 and d2), whose masses are between 15 and 18 kDa. They are very similar to the a and p type chains of vertebrates.

The second category, comprising 36 to 42 elements, groups together the so-called “structure” or “linker” polypeptide chains having little or no active site but allowing the assembly of subunits called twelfths or protomers. There are two types of linkers, L1 and L2.

Each extracellular hemoglobin molecule consists of two superimposed hexagons called hexagonal bilayer, wherein each hexagon is itself formed by the assembly of six subunits (dodecamer or protomer) in the form of a drop of ‘water. The native molecule is made up of twelve of these subunits (dodecamer or protomer). Each subunit has a molecular mass of about 250 kDa, and constitutes the functional unit of the native molecule.

Preferably, the extracellular hemoglobin of Annelids is chosen from among the extracellular hemoglobins of Annelid Polychetes and the extracellular hemoglobins of Annelid Oligochaetes. Preferably, the extracellular hemoglobin of Annelids is chosen from extracellular hemoglobins of the Lumbricidae family, extracellular hemoglobins of the Arenicolidae family, and extracellular hemoglobins of the Nereididae family. Even more preferably, the extracellular hemoglobin of Annelids is chosen from the extracellular hemoglobin of Lumbricus terrestris, the extracellular hemoglobin of Arenicola sp and the extracellular hemoglobin of Nereis sp, more preferably the extracellular hemoglobin of Arenicola marina or of Nereis virens. The arenicola Arenicola marina is a polychaete annelid worm living mainly in sand. More preferably the extracellular hemoglobin is the one of Arenicola marina; it is also called M101 in the present application.

Arenicola marina hemoglobin (Hb) is an extracellular soluble Hb sharing high homology with adult human hemoglobin HbA and with the particularity of being a giant and hexagonal-bilayer Hb (3600 kDa, 25×15 nm nanoparticle size) as compared to the 64 kDa of the HbA. It has the ability to fix 156 oxygen molecules (versus 4 for HbA) and to control the oxidative stress through a superoxide dismutase (SOD) activity based on copper and zinc. It is also called M101, and may be used as a clinical device supplemented in preservation solutions, in static cold storage and machine perfusion, in order to improve transplant preservation from an ischemia-reperfusion cycle, and with a breakthrough reported regarding graft recovery and long-term survival. Indeed, during organ transplantation, the period of ischemia leads to ATP depletion, acidosis, and reactive oxygen species (ROS) production, leading to cell death, complement activation and in turn to inflammation and immune activation.

According to the invention, the globin protomer of the extracellular hemoglobin of Annelids constitutes the functional unit of the native hemoglobin, as indicated above. Finally, the globin chain of the extracellular hemoglobin of Annelids may be chosen, in particular, from globin chains of the Ax and/or Bx type of extracellular hemoglobin of Annelids.

The extracellular hemoglobin of Annelids, its globin protomers and/or its globins do not require a cofactor to function, unlike the mammalian hemoglobin, in particular human. Finally, the extracellular hemoglobin of Annelids, its globin protomers and/or its globins having no blood typing, make it possible to avoid any problem of immunological reaction. The extracellular hemoglobin of Annelids, its globin protomers and/or its globins exhibit intrinsic SOD activity. Consequently, this intrinsic antioxidant activity does not require any antioxidant to function, unlike the use of a mammalian hemoglobin for which the antioxidant molecules are contained inside the red blood cell and are not linked to hemoglobin.

The extracellular hemoglobin of Annelids, its globin protomers and/or its globins may be native or recombinant.

According to the invention, the globin, the globin protomer or the extracellular hemoglobin of Annelids is preferably present in a composition comprising a buffer solution. According to the invention and as indicated in the examples, the globin, the globin protomer or the extracellular hemoglobin of Annelids, is preferably present (formulated) in a composition. Preferably, such a composition consists solely of a globin, a globin protomer or an extracellular hemoglobin of Annelids, and a buffer solution.

The formulation of the globin, the protomer of globin, or the extracellular hemoglobin of Annelids, in liquid form has the advantage of being more easily administered.

Said buffer solution creates an adequate salt environment for the transporter and, in particular, hemoglobin, its protomers and its globins, and thus allows the maintenance of the quaternary structure, and, therefore, of the functionality of this molecule. Thanks to the buffer solution, the transporter and, in particular, the hemoglobin, its protomers, and its globins are capable of ensuring their oxygenation function.

The buffer solution according to the invention is preferably an aqueous solution comprising salts, preferably chloride, sodium, calcium, magnesium and potassium ions, and gives the composition according to the invention a pH of between 6.5 and 7.6; its formulation is similar to that of a physiologically injectable liquid. Under these conditions, the extracellular hemoglobin of Annelids, its globin protomers, and its globins, remain functional.

In the present description, the pH is understood to be at ambient temperature (25° C.), unless otherwise stated.

Preferably, the buffer solution is an aqueous solution comprising sodium chloride, calcium chloride, magnesium chloride, potassium chloride, as well as sodium gluconate and sodium acetate, and has a pH of between 6.5 and 7.6, preferably equal to 7.1±0.5, preferably approximately 7.35. More preferably, the buffer solution is an aqueous solution comprising 90 mM NaCl, 23 Mm Na-gluconate, 2.5 mM CaCl2, 27 mM Na-acetate, 1.5 mM MgCl2, 5 mM KCl, and has a pH of 7.1±0.5, which may contain between 0 and 100 mM of antioxidant such as ascorbic acid and/or reduced glutathione.

The globin, the globin protomer or the extracellular hemoglobin of Annelids may be administered to the subject in a therapeutically effective amount. By “therapeutically effective amount” is meant an amount sufficient to achieve a concentration of compound which is capable of preventing or slowing down the disease to be treated. Such concentrations can be routinely determined by those of skilled in the art. The amount of the globin, globin protomer or extracellular hemoglobin of Annelids actually administered will typically be determined by a physician or a veterinarian, in the light of the relevant circumstances, including the condition to be treated, the chosen route of administration, the actual compound administered, the age, weight, and response of the patient, the severity of the subject's symptoms, and the like. It will also be appreciated by those of skilled in the art that the dosage may be dependent on the stability of the administered compound.

The globin, the globin protomer or the extracellular hemoglobin of Annelids may be administered by any means that achieve the intended purpose. For example, administration may be achieved by a number of different routes including, but not limited to subcutaneous, intravenous, intradermal, intramuscular, intraperitoneal, intracerebral, intrathecal, intranasal, oral, rectal, transdermal, buccal, topical, local, inhalant or subcutaneous use. Parenteral and topical routes are particularly preferred.

Dosages to be administered depend on individual needs, on the desired effect and the chosen route of administration. It is understood that the dosage administered will be dependent upon the age, sex, health, and weight of the recipient, concurrent treatment, if any, frequency of treatment, and the nature of the effect desired. The total dose required for each treatment may be administered by multiple doses or in a single dose. The doses used for the administration can be adapted as a function of various parameters, and in particular as a function of the mode of administration used, of the relevant pathology, or alternatively of the desired duration of treatment. For example, it is well within the skill of the art to start doses of the compounds at levels lower than those required to achieve the desired therapeutic effect and to gradually increase the dosage until the desired effect is achieved. However, the daily dosage may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 10 mg/kg of body weight per day.

The invention is described in more detail in the following examples. These examples are provided for illustration purposes only, and are not limitative.

### FIGURES

Legends of the figures are the following.

FIG. 1: M101 inhibits complement-dependent cytotoxicity crossmatch (CDC-XM) positivity.

Sorted blood B- and T-cells from organ donors were incubated with a negative AB control serum or a positive control serum (see Table 1 for details), and next with rabbit complement containing various concentrations of M101 (buffer, 1 g/L, or 5 g/L). Assessment between live and dead cells was achieved by fluorescent microscopy, and a lysis score was established (see material and methods). Results of 8 independent experiments and results are expressed as box plots of interquartile range. P values are indicated when significant (ANOVA test).

FIG. 2: Dose and time-effect of M101 on the 50% classical complement pathway (CH50).

EDTA whole blood from 6 healthy controls were incubated with increasing amounts of M101 (buffer, 0.25 g/L, 0.5 g/L, 1 g/L and 5 g/L) during 30 min, 2 h, 6 h, 12 h, 24 h and 48 h before being centrifuged and EDTA-plasma frozen (−80° C.). After that, CH50 activity was evaluated by hemolytic activity using IgG-coated sheep blood cells. A—Time effect. B—Dose effect, for each concentration all time points are indicated and results are expressed as box plots of interquartile range. P values are indicated when significant (p<0.05).

FIG. 3: No proteolytic action of M101 on C3 to C9 fractions.

EDTA whole blood from 6 healthy controls was incubated with increasing amounts of M101 (buffer, 0.5 g/L, 1 g/L and 5 g/L) during 12 h, 24 h and 48 h before being centrifuged and EDTA-plasma frozen (−80° C.). C3 and C4 fractions were quantified by turbidimetry, results are expressed as g/L, while radial immunodiffusion was used for the fractions C5 to C9 and results were expressed as the diameter of the ring (mm). Statistical analysis did not reach significance.

FIG. 4: M101 inhibits C3 convertase.

A-C: C3 convertase activation and capacity to cleave C3a (A) and downstream C5a (B) and sC5b9 (C) was evaluated following EDTA-plasma incubation with IgG-coated sheep blood cells. EDTA-plasma was obtained from EDTA whole blood incubated 6 h with an increasing amount of M101 (buffer, 0.25 g/L, 0.5 g/L, 1 g/L, and 5 g/L).

D-F: Capacity to release C3d in a luminex assay using 96 HLA class-I beads in the presence of M101 (buffer, 1 g/L and 5 g/L), complement fractions, and patient sera containing C3d positive anti-human leukocyte antigen (HLA)-I antibodies from one patient (D) or from a pool of patients (E: pool #1; F: pool #2). Results from the 96 HLA class-I beads are represented. The median fluorescence intensity (MFI) for each HLA class-I specificity is reported. P values are indicated when significant (p<0.05).

FIG. 5: Dose and time-effect of M101 on the alternative complement pathway (AP50).

Whole uncoagulated blood from 6 healthy controls were incubated with an increasing amount of M101 (buffer, 0.25 g/L, 0.5 g/L, 1 g/L and 5 g/L) during 30 min, 2 h, 6 h, 12 h, 24 h and 48 h before being centrifugated and serum frozen (−80° C.). AP50 activity was evaluated by hemolytic activity from sera using rabbit blood cells.

A—Time effect.

B—Dose effect, for each concentration all time points are indicated, and results are expressed as box plots of interquartile range. P values are indicated when significant (p<0.05).

FIG. 6: The classical and alternative complement pathways lead to the formation of C3 convertase (C4bC2a and C3bBb, respectively), which is inhibited by M101.

Downstream, C3 convertases cleave C3 into C3a (anaphylatoxin) and C3b. C3b amplifies the complement activation through formation of new C3 convertases and contributes to the formation of C5 convertases (C4b2a3b and C3bBb3b). C3 convertase is further inhibited by factors I and H that finally convert C3b into C3d. C5 convertases cleave C5 into the anaphylatoxin C5a and C5b to generate the membrane attack complex (MAC, C5bC6-C9) and the release of sC5b9. C3a and C5a possess anaphylatoxin properties, and C3d provides ligands for complement receptors (CR)2 present on B cells.

As a consequence, the use of M101 in transplantation may protect from antibody binding (classical pathway), alternative pathway activation, and subsequent effector functions.

FIG. 7: Circulating C3a concentration (ng/mL) in 4 dogs following M101 i.v. injection at TO (before test), T10 (10 min after injection), T60 (60 min), Texp (after extubation), T24 (24 h) and T48 (48 h).

As indicated, M101 was used at concentrations ranging from 0.5 mg/kg to 3.032 mg/kg. An ANOVA test was used for statistical analysis and the Tukey's test was used for multiple comparison tests.

### EXAMPLE 1: THE OXYGEN CARRIER M101 ALLEVIATES COMPLEMENT ACTIVATION BY INHIBITING THE C3 CONVERTASE

### Material and Methods

### Controls and Patients

Healthy staff members of the medical laboratory of the University Hospital of Toulouse (CHU de Toulouse, France) and of Brest (CHU Brest, France) were selected. Exclusion criteria, when known, included immune related diseases, and an active infection in the last 6 months. Peripheral blood was collected in uncoagulated and EDTA-anticoagulated tubes. The inventors further enrolled 8 kidney transplant recipients who had previously tested positive for anti-HLA Ab, and were used as positive controls in the routine complement-dependent cytotoxicity crossmatch assay (CDC-XM). Healthy individuals were volunteers, have given their informed consent, and according to the French law related to retrospective observational studies and our Institutional Review Board (University Hospital of Toulouse—Office of Research, Development and Innovation), the patient's need for written consent was waived.

### Complement-Dependent Cytotoxicity Crossmatch Assay

As previously described with adaptations (Milongo D, Vieu G, Blavy S, et al. Interference of therapeutic antibodies used in desensitization protocols on lymphocytotoxicity crossmatch results. Transpl Immunol. June 2015; 32(3):151-5), B and T cells were sorted using an EasySep® Kit (STEMCELL Technologies, Vancouver, Canada) from the buffycoat obtained from heparinized blood of living organ donors. Next, sorted B and T cells were incubated 30 min at 22° C. with: (i) heat-inactivated AB human serum used as a negative control (Sigma-Aldrich, Saint-Louis, Mo); (ii) heat-inactivated sera from patients containing human anti-leukocyte antigen (HLA) class I and/or II DSA; (iii) chimeric anti-HLA class-I human monoclonal IgG1 (Invivogen®, Toulouse, France); (iv) IgG anti-lymphocyte mAb (One Lambda, Thermo-Fisher, Canoga Park, CA); or (iv) IgM anti-human 32 microglobulin mAb (BD biosciences, San Jose, CA). Rabbit complement (Servibio, Meudon, France) preincubated up to 8 h with increasing amounts of M101 (extracellular hemoglobin of Arenicola marina) (buffer alone, 1 g/L, and 5 g/L; HEMO₂Life®, Hemarina, Morlaix, France) was then added in Terasaki plates (Greiner bio-one, Les Ulis, France) for 60 min at 22° C. Separation between live (colored in green) and dead cells (colored in red) was achieved by fluorescent microscopy, using the FluoroQuench® staining/quenching reagent (One Lambda). Results were scored as follows: score 1: 1-10% dead cells; score 2: 11-20%; score 4: 21-50%; score 6: 51-80%; and score 8: 81-100%.

### C3d Single-HLA Antigen Bead Assay

The Lifecodes® C3d single-antigen Luminex based assay (Immucor, Stamford, CT) was adapted as follows: single antigen beads (40 μL) were incubated 30 min at 22° C. with 10 μL of heat-inactivated serum from a patient containing anti-HLA class I Ab or from a pool of human anti-HLA class I Ab (Immucor, Ref: LSAPC1). Next were added 30 μL of a solution containing human complement fractions (CD3dCS, Immucor) and previously were pre-incubated 1 h with 10 μL of increasing amount of M101 (buffer alone, 1 g/L and 5 g/L final concentration). After 30 min at 22° C., and several wash steps, a phycoerythrin coupled anti-C3d mAb was added in order to recognize the C3d fraction bound to the beads in response to the classical complement pathway activation. Finally, the median fluorescence intensity (MFI) for each HLA class-I bead was determined on a luminex 200 reader (Luminex Corporation, Austin, TX) and analyzed with MatchIT software (Immucor).

### CH50 and AP50

Fixed volumes of freshly collected EDTA-anticoagulated blood for CH50 or serum derived from freshly uncoagulated blood for AP50 were incubated 30 min, 2 h, 6 h, 12 h, 24 h and 48 h at room temperature with increasing amounts of M101 (buffer alone, 0.25 g/L, 0.5 g/L, 1 g/L, and 5 g/L final concentration). Plasma and sera were collected after centrifugation, and frozen immediately at −80° C. The kinetic hemolytic assays for quantification of classical and alternative pathways were previously described (Puissant-Lubrano B, Fortenfant F, Winterton P, Blancher A. A microplate assay to measure classical and alternative complement activity. Clin Chem Lab Med. May 1, 2017; 55(6):845-853; Puissant-Lubrano B, Puissochet S, Congy-Jolivet N, et al. Alternative complement pathway hemolytic assays reveal incomplete complement blockade in patients treated with eculizumab. Clin Immunol. October 2017; 183:1-7). Briefly, for CH50, EDTA-plasma was added to IgG sensitized sheep red blood cells in the presence of 15 mmol CaCl₂, and for AP50 sera was added to rabbit erythrocytes on microplates to assess a continuous measure of the optical density at 540 nm by means of a spectrophotometer microplate reader (iEMS reader/dispenser; ThermoFisher, Beverly, MA). Complement activity was deduced from the calibration range and expressed as a percentage of the lysis produced with the calibrator. In selected experiments, CH50 supernatants from the 6 h dose-response effect of M101 on EDTA-plasma or from 120 μg/mL eculizumab treated EDTA-plasma (Soliris; Alexion Pharmaceuticals, Inc., Boston, MA) were recovered after 7 min on IgG sensitized sheep red blood cells, centrifuged and used immediately or frozen at −80° C. until C3a, C5a and sC5b9 quantification.

### Complement Fractions

EDTA whole blood was incubated with M101 (buffer, 0.5 g/L, 1 g/L, and 5 g/L) at different time points (12 h, 24 h, and 48 h) and complement fractions were quantified in collected EDTA-plasma by turbidimetry (Cobas 8000, Roche, Meylan, France) for complement fractions C3 and C4, and by radial immunodiffusion (The Binding Site, Birmingham, UK) for the fractions C5 to C9. Results were expressed as g/L for C3 and C4 according to manufacturer's instructions, and as the diameter of the ring (mm) for C5 to C9.

C3a, C5a and sC5b-9 were quantified from CH50 supernatants (see above) by ELISA (MicroVue PLus EIA, Quidel, distributed by Eurobio-Scientific Les Ullis, France), according to the manufacturer's instructions.

### Statistical Analysis

Continuous data are described as median and interquartile range (IQR) 25^{th}-75^{th} percentile for non-parametric analysis and as mean±standard error of the mean (SEM) for parametric analysis. Differences between groups were analyzed using a paired multiple one-way ANOVA, and the Tukey's test was used for post hoc comparison. Data were analyzed using GraphPad Prism 9.2 (La Jolla, CA), and a p<0.05 considered as significant.

### Results

### M101 Inhibits Crossmatch Positivity

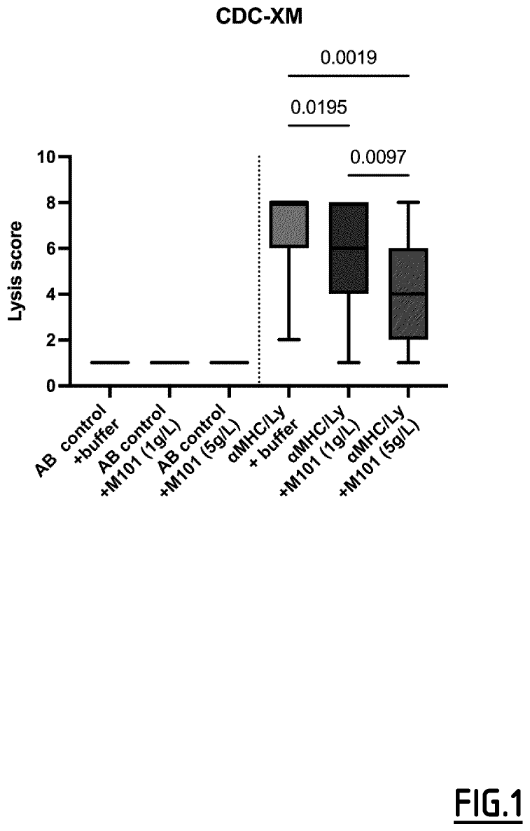

Among patients waiting for transplantation at the university hospital center of Toulouse, up to 34% have pre-formed antibodies to HLA class I/II (CB and NC, personal data) raising the question whether M101, which can be used in organ preservation solutions in transplantation, affects Ab mediated CDC-XM assay using purified T cells (HLA class 1) or B cells (HLA class I and II), and rabbit sera as source of complement. Such analysis summarized in Table 1 and presented in FIG. 1 revealed that: (i) M101 when used at 1 g/L and 5 g/L did not exert a direct cytotoxic effect on T/B cells in the presence of the negative AB human sera control (lysis score=1, FIG. 1 left); (ii) M101 was effective in affecting CDC-XM when used at 1 g/L (median lysis score=6.0 [IQR: 4.0-8.0] versus 8.0 [6.0-8.0] with buffer; p=0.02) and even more at 5 g/L (median lysis score=4.0 [2.0-6.0]; p=0.002) (FIG. 1 right); (iii) the inhibitory effect was retrieved in both T and B cells; and (iv) the inhibitory effect was independent of the antigenic target as effective with anti-HLA, anti-human (32 microglobulin, and anti-lymphocyte mAbs. Altogether, these results support that M101 was effective in reversing at least partially the rabbit complement-dependent cytotoxic effect mediated by the binding of the antibodies in the CDC-XM assay.

TABLE 1
Crossmatch complement dependent lysis score obtained
from 8 independent experiments. B and T cells were
purified from peripheral blood human living donors.

| | | Cell | | M101 | M101 |
|---|---|---|---|---|---|
| Experiment | Abs | type | Buffer | (1 g/L) | (5 g/L) |
| #1 | AB control | T cells | 1 | 1 | 1 |
| #1 | anti-hLy | T cells | 8 | 6 | 2 |
| #2 | AB control | B cells | 1 | 1 | 1 |
| #2 | DSA-I | B cells | 8 | 6 | 6 |
| #2′ | AB control | T cells | 1 | 1 | 1 |
| #2′ | DSA-I | T cells | 8 | 8 | 6 |
| #3 | AB control | B cells | 1 | 1 | 1 |
| #3 | DSA-II | B cells | 6 | 4 | 4 |
| #4 | DSA-I | B cells | 8 | 8 | 8 |
| #4 | anti-hLy | B cells | 6 | 6 | 4 |
| #5 | AB control | B cells | 1 | 1 | 1 |
| #5 | xAb-I | B cells | 8 | 6 | 4 |
| #6 | AB control | B cells | 1 | 1 | 1 |
| #6 | anti-hLy | B cells | 8 | 8 | 6 |
| #7 | AB control | T cells | 1 | 1 | 1 |
| #7 | xAb-I | T cells | 6 | 4 | 1 |
| #8 | AB control | T cells | 1 | 1 | 1 |
| #8 | DSA-I | T cells | 2 | 1 | 1 |
| #8 | anti-hβ2 | T cells | 8 | 8 | 6 |

Abbreviations: Abs: antibodies; AB control: AB human serum used as negative control; DSA-I: donor specific Abs anti-HLA class I; xAb-I: chimeric anti-HLA monoclonal antibody targeting anti-human HLA class I public epitopes (500 ng/mL); Anti-hβ2: IgM anti-human β2 microglobulin; and anti-hLy: IgG anti-human lymphocytes (target unknown).

### M101 Inhibits the Classical Pathway in a Dose-Dependent Manner

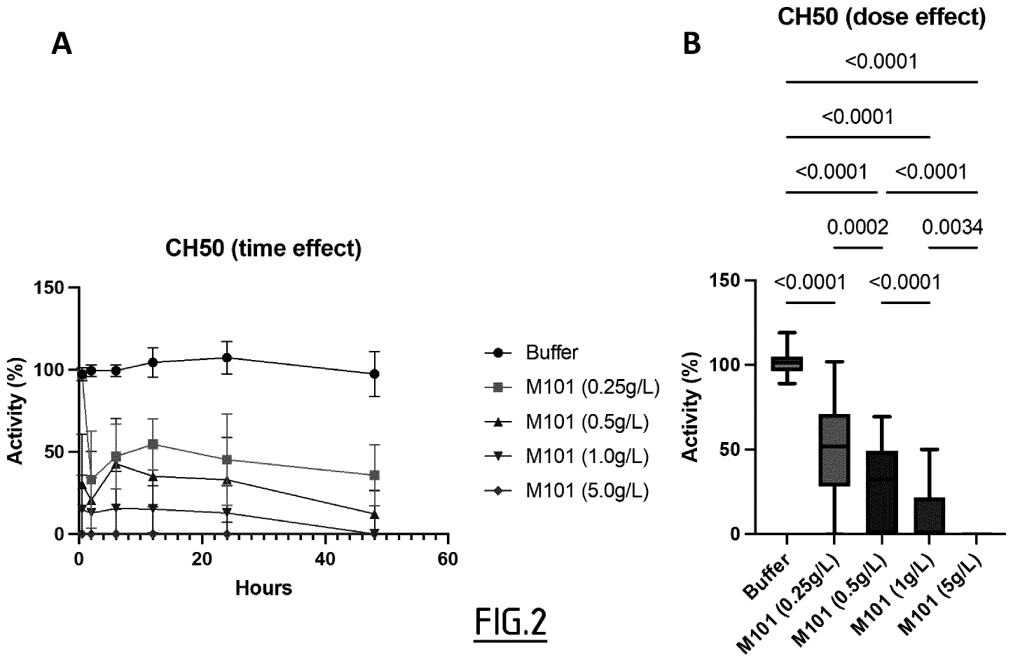

To go further in the exploration of the M101 capacity to interfere with the CDC-XM, EDTA whole blood from 6 healthy individuals was incubated with increasing amounts of M101 (buffer, 0.25 g/L, 0.5 g/L, 1 g/L and 5 g/L), and then EDTA-plasma was collected at six time points (30 min, 2 h, 6 h, 12 h, 24 h, and 48 h). Next, the time effect of M101 on the complement classical pathway (CH50) using IgG sensitized sheep erythrocytes was conducted. As presented in FIG. 2A, the M101 capacity to prevent CH50 activation was achieved at the first time point (30 min) and remained stable until 48 h. Compiling results retrieved a dose effect with complete inhibition reported when using M101 at 5 g/L (p<10⁻⁴), while activity was almost complete at 1 g/L (0.0% [IQR: 0.0-21.5%]; p<10⁻⁴), and partial at 0.5 g/L (32.6% [0.0-49.3%]; p<10⁻⁴) and 0.25 g/L (52.0% [28.3-71.0%]; p<10⁻⁴) (FIG. 2B). Indeed, these findings demonstrated that the activation of the classical complement pathway in the CH50 assay was controlled in a dose-dependent and stable manner by M101.

M101 does not Exert a Proteolytic Effect on Complement Fractions

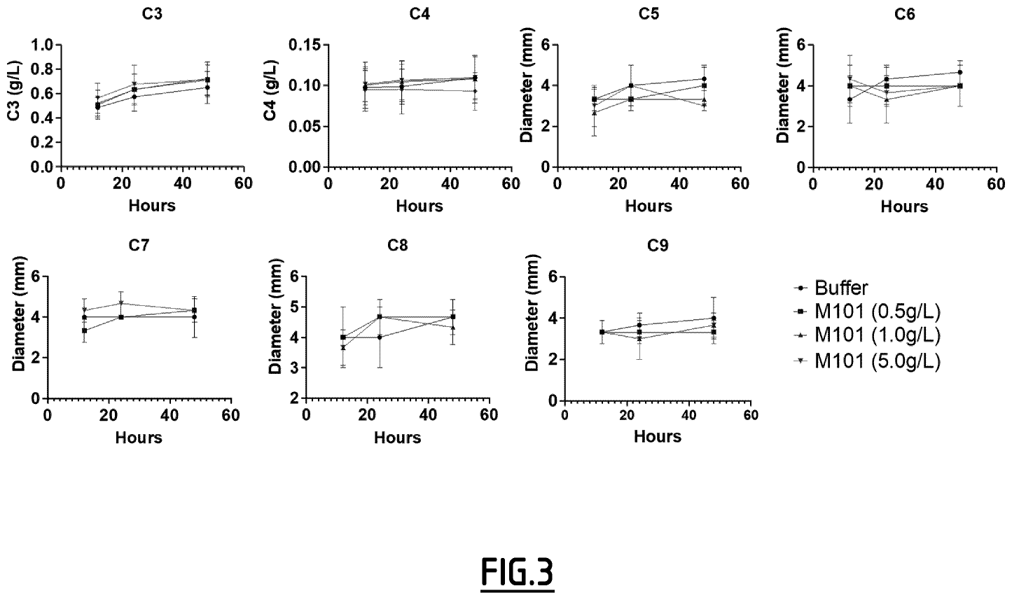

To test the proteolytic effect of M101 on proteins involved in the classical pathway, EDTA whole blood from healthy individuals was pre-incubated with increasing amounts of M101 (buffer, 0.5 g/L, 1 g/L and 5 g/L) and centrifuged at three time points (12 h, 24 h, and 48 h). Next, EDTA-plasma containing complement fractions was evaluated by turbidimetry (C3 and C4; n=10) or radial immunodiffusion (C5, C6, C7, C8, and C9; n=3). As presented in FIG. 3, C3-C9 fraction levels were stable over time and not affected by addition of increasing amounts of M101. Altogether, these data excluded the possibility that M101 exerted a proteolytic effect on the complement fractions C3-C9. The inventors then postulated that the inhibitory effect of M101 functioned through interaction with the complement macromolecular complexes.

### M101 as an Inhibitor of C3 Mediated Complement Activation

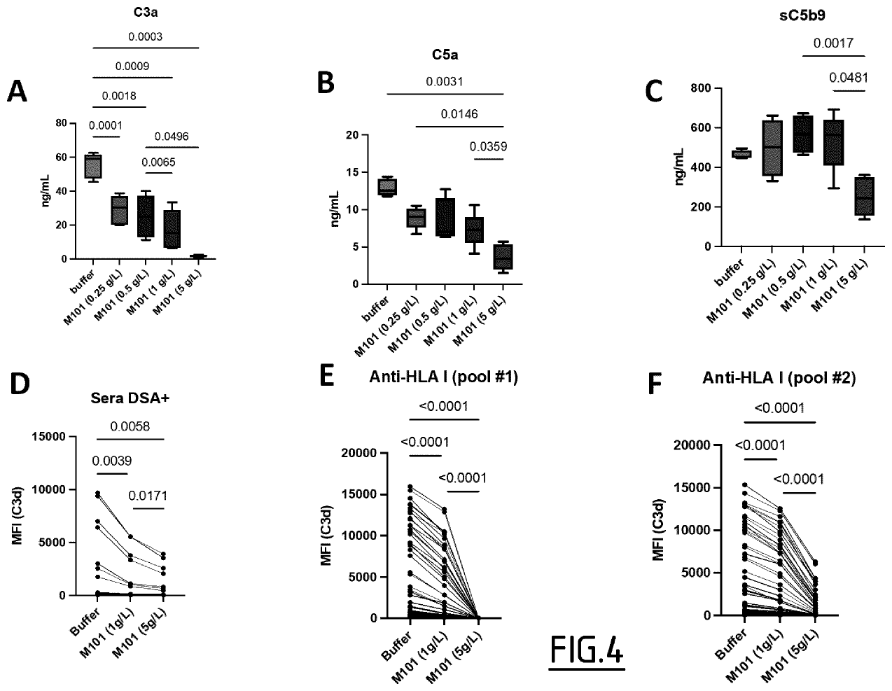

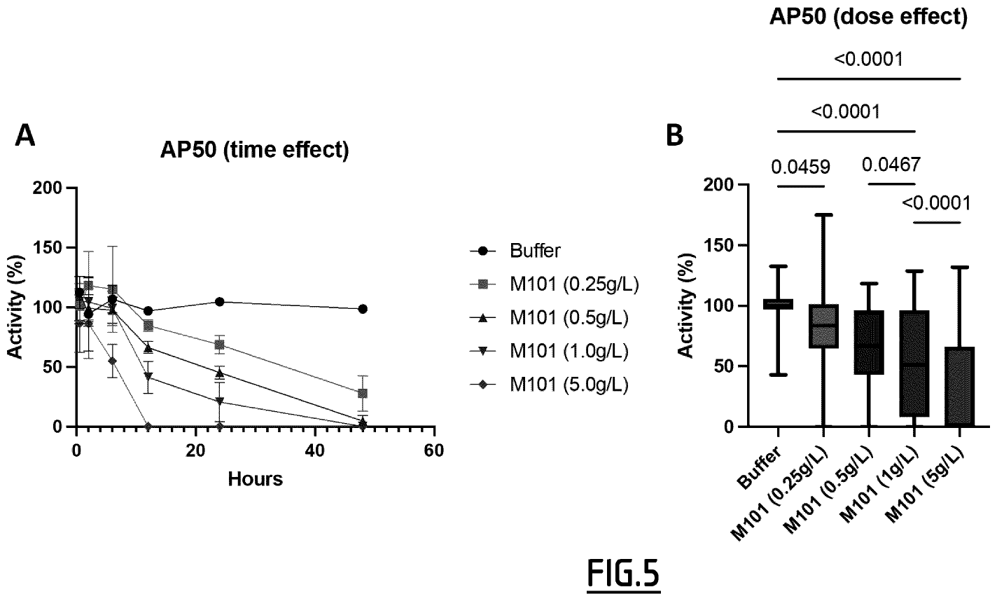

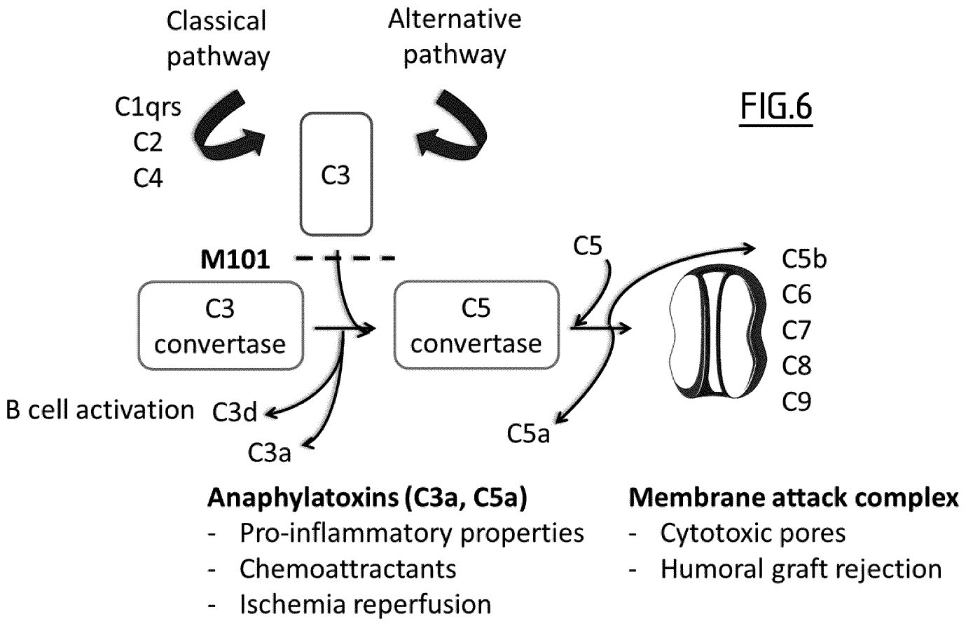

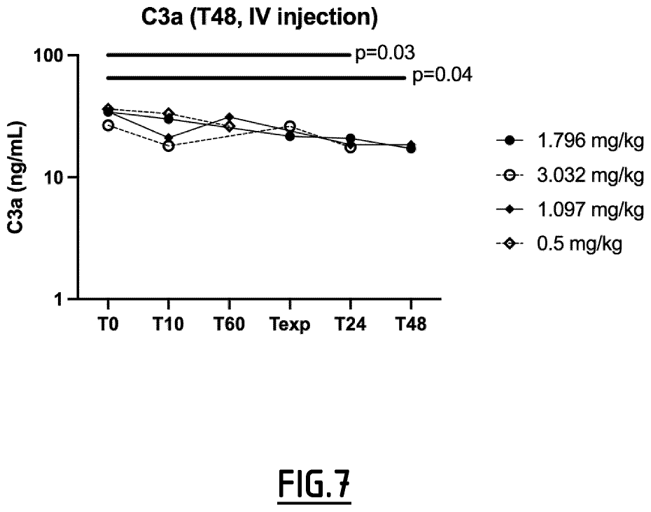

Complement common pathway activation involves macromolecular complexes that sequentially cleave C3 to C3a and C3b (C3 convertases), C5 to C5a and C5b (C5 convertases), and contribute to MAC formation with the release of the soluble terminal complement complex (sC5b9). In order to test these macromolecular complexes, M101-treated plasma selected from 5 healthy donors was incubated with IgG sensitized sheep red blood cells (CH50 assay) and the release of C3a, C5a and sC5b9 was next quantified. Compared with the anti-human C5 mAb (Eculizumab) that profoundly inhibited C5a and sC5b-9 generation but had no effect on C3a generation (data not shown), M101 significantly inhibited the production of C3a, C5a and sC5b9 (FIG. 4A/C). Again, a dose effect was observed with M101 and the maximal effect was retrieved at 5 g/L as this concentration was effective in impairing the formation of C3a (p=0.0003), C5a (p=0.003), and sC5b9 (p=0.002 versus M101 0.5 g/L).

C3 convertase converts C3 in C3a and C3b, and the degradation and inactivation of C3b by the factors I and H lead to the release of C3d. Accordingly, the M101 capacity to inhibit the release of C3d in a luminex assay with single-HLA class I antigen beads was tested using patient sera containing anti-HLA class-I Abs from one patient or from a pool of patients from the commercial assay, the latter being tested twice (FIG. 4 D-F). M101 was effective in reducing the MFI of anti-C3d staining when used at 1 g/L (0.0004<p<10⁻⁴) and 5 g/L (0.006<p<10⁻⁴). Of note, the M101 inhibitory effect was not restricted to the anti-HLA Ab target antigen (MFI positive cut-off>1500), but was retrieved for all the 96 HLA class-I antigens (MFI range: 56-15952). Altogether, this suggests an action of M101 on the C3 convertase, which is common with the alternative pathway, or upstream (C1qrs, C2 and C4).

### M101 Exerts its Inhibitory Effect on C3 Convertase

Finally, M101 capacity to inhibit the alternative pathway was evaluated as this pathway starts with the formation of the C3 convertase in a process independent from Ab mediated C1qrs, C2 and C4 activation. To this end, sera from 5-6 healthy controls were incubated with increasing amounts of M101 (buffer, 0.25 g/L, 0.5 g/L, 1 g/L, and 5 g/L) at different time points (30 min, 2 h, 6 h, 12 h, 24 h, and 48 h). By using these sera, time and dose effects of M101 were evaluated with the alternative pathway method (AP50), which evaluated lysis of rabbit erythrocytes in the presence of Mg⁺². As presented FIG. 5A, the maximal inhibitory effect of M101 on AP50 was delayed to 12 h when using M101 at 5 g/L, to 48 h when using M101 at 1 g/L and 0.5 g/L, and not achieved after 48 h with M101 at 0.25 g/L. When compiling all data (FIG. 5B), a dose effect was observed with the lower activity reported with M101 at 5 g/L (0.0% [IQR: 0.0-66.3%], p<10⁻⁴ versus buffer) as compared with M101 at 1 g/L (51.0% [8.0-96.3%], p<10⁻⁴), 0.5 g/L (67.0% [43.0-96.1%], p<10⁻⁴), and 0.25 g/L (83.7% [64.5-100%], p=0.05). Altogether, this confirms the capacity of M101 to control C3 convertase activation and amplification in a dose-dependent manner.

### CONCLUSION

Complement activation constitutes a cornerstone in transplantation, including in allogeneic antibody mediated rejection through the classical pathway and ischemia/reperfusion through the tissue damage capacity to mediate the alternative pathway. As a consequence, taming these pathways is of utmost interest and the description of a direct blockade exerted on the complement cascade by M101 (FIG. 6) coupled with organ preservation, to reduce complement activation, provide an explanation for the shortened time for graft recovery and long-term survival associated with the use of M101 as an oxygen carrier in the organ preservation solution.

The deleterious effect on grafts of allogeneic anti-HLA Abs through complement activation is well established, and new therapeutic strategies are needed to prevent/cure this rejection. Indeed, animal models have shown that early blockade of the common pathway is effective in preventing acute antibody-mediated rejection, and when used in transplant recipients with preformed DSA the humanized anti-human C5 monoclonal antibody, eculizumab, prevents in part antibody-mediated kidney rejection. Similar to eculizumab, M101 when used at therapeutic and subtherapeutic concentrations (1-5 g/L) was effective in inhibiting the cleavage of C5 to C5a and C5b, the formation of sC5b9, and complement-mediated lysis in the CH50/AP50 assays. However, and in addition to eculizumab, M101 inhibits the complement cascade early in the cascade and thereby prevents the cleavage of C3 into C3a and downstream C3d. Moreover, such an effect is not human specific as retrieved in the CDC-XM assay, which uses rabbit sera as the source of complement fractions. Rabbit and human complement activation present differences, which may explain the partial effect reported in the CDC-XM assay as compared to the CH50 and AP50 assays. Of note and not tested in this study, the amino sequence of the C3 ortholog from annelid is 29% identical with its human counterpart, supporting a role for M101 to regulate in vivo the level of C3 convertase, and in turn complement pathway activation, in Arenicola marina.

As shown in a previous clinical trial, M101, when used in preservation solution in renal transplantation, prevents delayed graft function (DFG), a signature of ischemia/reperfusion injury. The role of M101 on ischemia/reperfusion is further supported by the observations that M101 was effective in a dose-dependent manner to block the release of the anaphylatoxins C3a and C5a, an inhibition that is critical to prevent ischemia/reperfusion injury as reported within the C3aR/C5aR knock-down model. Indeed, anaphylatoxins are potent mediators of inflammation and exert their innate and adaptive functions via their cognate receptors present on phagocytic cells (e.g., neutrophils and macrophages) and T/B cells. M101 also leads to a decrease in C3d deposition from anti-HLA DSA as demonstrated when using a graft immunocomplex capture fluorescence analysis technique. Altogether these results indicate that M101 allows the preservation of allograft organs from complement-mediated injury, which can be achieved by two ways: one indirect through maintaining the aerobic metabolism therefore avoiding the activation of the complement, and one direct through a direct inhibition exerted on the C3 convertase. These results bring an explanation of the benefits brought by M101 not only to preserve graft quality but also to avoid inflammation and transplant rejection, which may prevent subsequent use of complement pathway inhibitors such as eculizumab.

C3-targeted inhibitors and their transfer to the clinic are considered as promising. Indeed, C3 specific inhibitors using peptides and nanobodies platforms have been developed, with clinical trials conducted in a large panel of diseases including periodontal inflammation and COVID-19 with AMY-101, in paroxysmal nocturnal haemoglobinuria with pegcetacoplan, and in acute antibody-mediated graft injury and age-related macular degeneration with Cp40. In addition to its anti-complement properties, M101 has original characteristics including oxygen carrier, anti-inflammatory, anti-bacterial and superoxide dismutase antioxidant properties. It is further notable that no immunological, allergic or prothrombotic effects were associated with M101 injection in mice, and, in contrast to cell-free HbA, M101 did not lead to hypertension, myocardial infarction, renal damage, complement activation or tissue toxicity, which supports systemic usage. All these properties open new perspectives for M101 to treat ischemia pathologies inducing hypoxia, production of ROS, complement activation and inflammation.

In conclusion, the inventors have presented here that M101 inhibits the CDC-XM, classical and alternative complement pathways, and anaphylatoxin C3a and C5a releases. Such effects most probably rely on the capacity of M101 to avoid activation of the complement system when used at therapeutic concentrations (1-5 g/L). Importantly, these effects coupled with the exceptional properties of M101 (e.g; anti-inflammatory, antioxidant, non-immunological) open new therapeutic perspectives including in transplantation to prevent ischemia/reperfusion and allogenic humoral rejection, as well as in other complement-activation associated diseases.

### EXAMPLE 2: SYSTEMIC EFFECT OF M101 ON COMPLEMENT FRACTION C3a IN BEAGLE DOGS

### Material and Methods

In an attempt to determine whether systemic injection of M101 affects in vivo the endogenous activation of the complement pathway, 4 dogs (Beagles) were selected.

At 29-32 months old, dogs were exposed to M101 using intravenous injection (IV) in its liquid form: dog 1 (M101: 1.097 mg/kg); dog 2 (0.5 mg/kg); dog 3 (1.796 mg/kg); and dog 4 (3.032 mg/kg).

Peripheral blood plasma was collected at TO (before test), T10 (10 min after injection), T60 (60 min), Texp (after extubation), T24 (24 h) and T48 (48 h). Finally, the complement split product C3a (abx150230, Abbexa, Cambridge, UK) was tested and expressed in ng/mL according to the manufacturer's instructions.

### Results

Following M101 injection, the endogenous and basal level of C3a decreased (median: 34.5 ng/mL (IQR: 28.6-35.8)) at TO versus 18.5 ng/mL (IQR: 17.6-20.8) at T24 and 17.8 ng/mL (IQR: 17-18.5) at T48 (p=0.03 and 0.04, respectively).

This time effect was independent from M101 concentrations when using M101≥0.5 mg/kg. Altogether this supports that M101 is effective to prevent endogenous C3a split product release from C3 complement fraction in vivo.

1. Method for treating and/or preventing at least one complement-activation associated disease in a subject, comprising administering at least one molecule chosen from a globin, a globin protomer or an extracellular hemoglobin of Annelids to said subject.

2. The method according to claim 1, wherein it is a C3 convertase inhibitor, preferably an inhibitor of C3bBb and/or C4bC2a.

3. The method according to claim 1, wherein the complement-activation associated disease is chosen from diseases involving ischemia such as ischemia/reperfusion injury; immune and autoimmune disorders; renal diseases; acute and chronic inflammation dysfunctions; age-related and degenerative diseases; pregnancy-related complications; and hemolytic disorders.

4. The method according to claim 1, wherein the complement-activation associated disease is chosen from ischemia/reperfusion injury, transplant rejection such as antibody-mediated rejection, heart and vascular diseases (for example ischemia/myocardial infarction), cerebral and intestine embolism (for example stroke or mesenteric ischemia); systemic lupus erythematosus, anti-neutrophil cytoplasmic antibody (ANCA)-associated vasculitis and Crohn's disease; C3 glomerulopathies, atypical hemolytic-uremic syndrome and other thrombotic microangiopathies; COVID-19 and other viral infections, hemodialysis, implants, sepsis and cancer; Alzheimer's syndrome, schizophrenia and macular degeneration; preeclampsia; and paroxysmal nocturnal hemoglobinuria.

5. The method according to claim 1, wherein the complement-activation associated disease is chosen from ischemia/reperfusion injury and transplant rejection.

6. The method according to claim 1, wherein the complement-activation associated disease is transplant rejection.

7. The method according to claim 4, wherein the transplant is a kidney, a heart, an intestine, a pancreas, a liver or a lung, preferably it is a kidney.

8. The method according to claim 4, wherein the transplant rejection is an acute transplant rejection or a chronic transplant rejection.

9. The method according to claim 4, wherein the transplant rejection is an antibody-mediated rejection or a T cell-mediated rejection, preferably an antibody-mediated rejection.

10. The method according to claim 1, wherein it is an extracellular hemoglobin of Annelids chosen from among the extracellular hemoglobins of Annelid Polychetes and the extracellular hemoglobins of Annelid Oligochaetes.

11. The method according to claim 1, wherein it is chosen from extracellular hemoglobins of the Lumbricidae family, extracellular hemoglobins of the Arenicolidae family and extracellular hemoglobins of the Nereididae family, preferably the extracellular hemoglobin of Annelids is chosen from the extracellular hemoglobin of Lumbricus terrestris, the extracellular hemoglobin of Arenicola sp and the extracellular hemoglobin of Nereis sp, more preferably the extracellular hemoglobin of Arenicola marina or of Nereis virens.

12. The method according to claim 1, wherein it is the extracellular hemoglobin of Arenicola marina.

13. The method according to claim 1, wherein it is formulated in a composition comprising a buffer solution.

14. The method according to claim 13, wherein the buffer solution is an aqueous solution comprising salts, preferably chloride, sodium, calcium, magnesium and potassium ions, and gives the composition a pH of between 6.5 and 7.6.
